(19) United States
(12) Patent Application Publication
(10) Pub. No.: US 2004/0073117 A1
(43) Pub. Date: Apr. 15, 2004

(54) Method and system for delivering contrast agent

(76) Inventors: Karl Q. Schwarz, Canadiagua, NY (US); Xucai Chen, Rochester, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/250,461

(22) PCT Filed: Feb. 14, 2002
(86) PCT No.: PCT/US02/04770

(51) Int. Cl. A61B 8/14

(52) U.S. Cl. 600/458

(57) ABSTRACT

A contrast effect measuring unit 104 measures the contrast effect of an ultrasound contrast agent in a patient 110. The control unit 106 determines a desired contrast agent infusion rate, based on the contrast effect measured by the contrast effect measuring unit 104 and programmed contrast effect parameters. The control unit 106 controls an infusion unit 102 to infuse the patient 110 with the determined infusion rate. An ultrasound imaging unit 108 performs an ultrasound imaging test on the patient 110. This process is continued through the course of the ultrasound imaging test, to maintain a steady contrast effect and a steady signal enhancement level for the patient 110.

This application claims priority to U.S. provisional application No. 60/268,814 filed on Feb. 14, 2001 and titled “Method and Device for the Delivery and Control of Ultrasound Contrast Agents.”

### BACKGROUND OF THE INVENTION

1. Field of the Invention

This application relates to the delivery and control of ultrasound contrast agent.

2. Description of the Related Art

Ultrasound imaging is a popular means of observing the physiological conditions of human or animal patients. Echo contrast is often employed to improve the quality of ultrasound images. Studies have indicated that echocardiography using contrast agent delivered as an infusion can achieve optimized signal enhancement with a desired contrast effect for a longer duration than bolus injection. In a contrast infusion, contrast agent is infused intravenously or intra-arterially into a human or animal patient to change the intravascular concentration of micro-bubbles in a patient. Contrast agents such as Optison® made by Mallinckrodt of St Louis, Mo. and Definity® made by Dupont of Wilmington, Del. are FDA approved for commercial use.

It is important to determine the proper contrast agent infusion rate, so that the proper micro-bubble concentration can be achieved and maintained during an ultrasound imaging measurement. If a contrast agent is infused too rapidly into a patient, then the intravascular compartment becomes too echogenic and makes the ultrasound images difficult to read. Infusing too much of a contrast agent also wastes the contrast agent. On the other hand, if not enough contrast agent is infused into the patient, then it is too echolucent and the ultrasound contrast effect is lost. The micro-bubble concentration of a contrast agent often varies from patient to patient, making it difficult to determine a proper contrast agent infusion rate.

Even if proper micro-bubble concentration is achieved at one point during the ultrasound imaging process, the micro-bubble concentration may fluctuate during the course of an ultrasound imaging process, thus limiting the duration of optimized signal enhancement. For example, even if a contrast agent is infused at a constant rate during the course of a diagnostic ultrasound to assess tissue perfusion, the micro-bubble concentration of the patient may fluctuate, leading to errors in tissue perfusion measurements. These conditions make it difficult to fix the proper contrast agent infusion rate during the course of an ultrasound imaging process. In addition, ultrasound imaging users cannot select their desired degree of contrast effect, which relates to the degree of signal enhancement. Currently, a user can only manually set and adjust the infusion rate based on intuition, experience and luck, hoping to achieve the desired degree of contrast effect.

### SUMMARY OF THE INVENTION

The present application discloses methods and systems for the delivery and control of contrast agent. The disclosed methods and systems can continuously measure a patient's intravascular contrast effect, and automatically adjust the infusion rate during an ultrasound imaging process. The disclosed methods and systems can automatically maintain a relatively steady micro-bubble concentration during the ultrasound imaging process, which is desirable for all types of ultrasound imaging and especially for assessing tissue perfusion. The disclosed methods and systems can produce a prolonged duration of ideal contrast effect and signal enhancement. The disclosed methods and systems allow a user to select a preferred degree of ultrasound contrast effect, which is desirable for all types of ultrasound imaging and especially for myocardial perfusion imaging.

One aspect relates to a method of determining an infusion rate for delivery of contrast agent for ultrasound imaging. The method includes measuring a contrast effect of a contrast agent in a patient, and automatically determining a contrast agent infusion rate based on the measured contrast effect. An ultrasound imaging test can then be performed on the patient with infusion of the contrast agent at the determined rate.

Another aspect relates to a system of controlling the delivery of contrast agent for ultrasound imaging. The system includes a contrast effect measuring unit configured to measure the contrast effect of a contrast agent in a patient, a control unit configured to determine a contrast agent infusion rate based on the measured contrast effect, and an infusion unit configured to infuse the patient with contrast agent at the determined contrast agent infusion rate.

Other aspects are described below in the application.

### BRIEF DESCRIPTION OF THE DRAWINGS

FIG. 1 shows one embodiment of a collection of units involved in the delivery and control of contrast agent.

FIG. 2 shows one embodiment of a contrast effect measuring unit.

FIG. 3 shows one embodiment of a control system.

FIG. 4 shows one embodiment of a programming interface for programming parameters.

FIG. 5 shows one embodiment of an ultrasound system.

FIG. 6 shows one embodiment of an ultrasound system and one embodiment of an infusion system.

FIG. 7 shows one embodiment of a process of delivering and controlling contrast agent.

### DETAILED DESCRIPTION OF THE PREFERRED EMBODIMENTS

For purposes of summarizing the invention, certain aspects, advantages and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

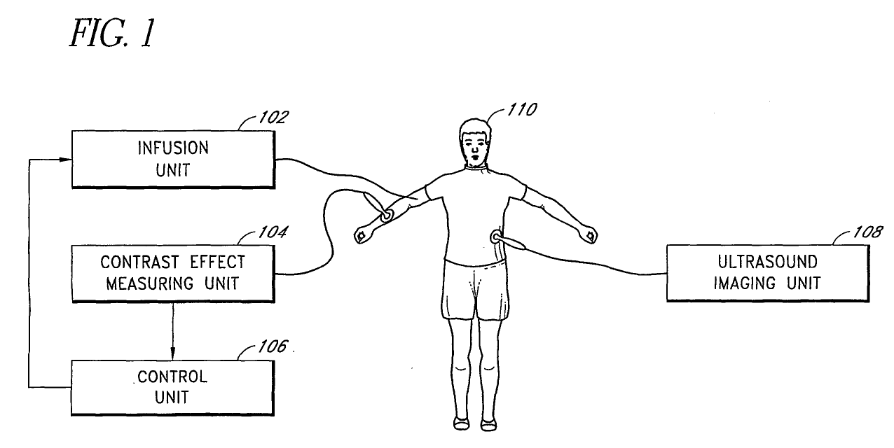

FIG. 1 shows one embodiment of a collection of units involved in the delivery and control of contrast agent. In the embodiment shown in FIG. 1, a contrast effect measuring unit 104 is connected to a human or animal patient 110 to measure the contrast effect of a contrast agent in the patient 110. In one embodiment, the measuring unit 104 is connected to a peripheral artery of the patient 110. For example, an ultrasound probe can be fixed to an arm or a leg of the patient 110. The probe preferably remains fixed to the same location during the course of an ultrasound imaging process to allow for continuous measuring. In one embodiment, the contrast effect measuring unit 104 measures the Doppler audio signal of the patient's blood vessel. In another embodiment, the measuring unit 104 measures contrast effect by measuring ultrasound signals. Thus, the contrast effect measuring unit 104 can include ultrasound, Doppler, or both measuring modalities.

The contrast effect measuring unit 104 receives measurements of the contrast effect and sends the measurements to a control unit 106. In one embodiment, the measuring unit 104 sends the raw measurements such as unprocessed Doppler audio signals of the patient's blood flow to the control unit 106. In another embodiment, the measuring unit 104 processes the measurements and sends the processed measurements to the control unit 106. Examples of processing the measurements include vector summation, RMS-DC (root mean square-direct current) conversion, logarithmic conversion, signal scaling, analog-digital conversion, and others.

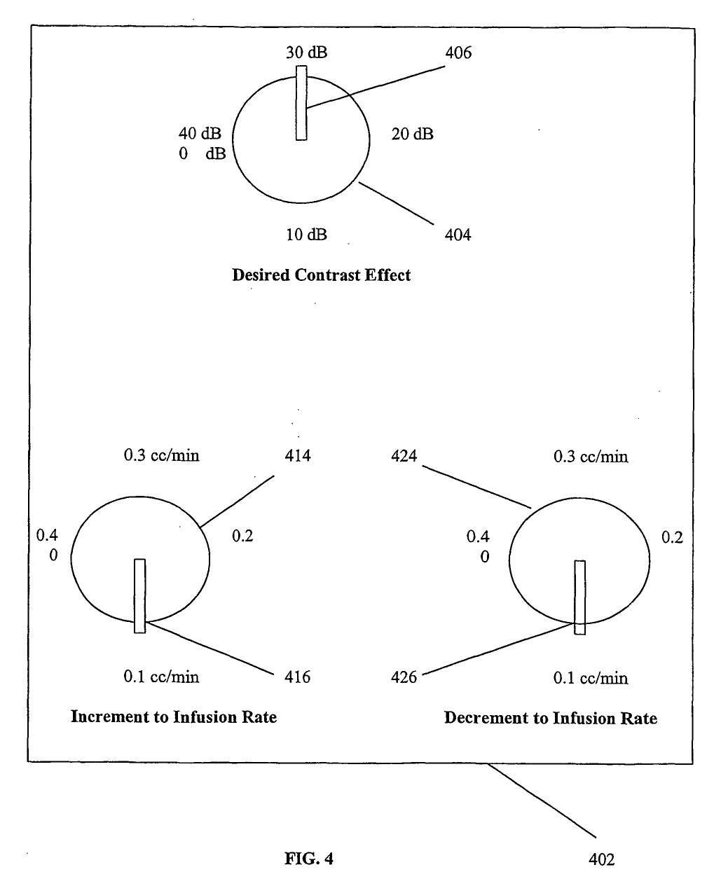

The control unit 106 can in some embodiments include a programming interface (not shown). A user administering an ultrasound imaging test uses the programming interface to set parameters. Parameters may include the initial contrast agent infusion rate (Rᵢₙᵢₜ), the desired contrast effect (C_{target}), the increase to the current infusion rate in the event that the measured contrast effect is lower than the desired contrast effect (R_{inc}), and the decrease to the current infusion rate in the event that the measured contrast effect is higher than the desired contrast effect (R_{dec}). For example, the desired contrast effect can be 30 dB (corresponding to audio signal intensity), the initial infusion rate can be 10-cc/minute, R_{inc} and R_{dec} can be 0.2-cc/minute. One embodiment of the programming interface is described below in connection with FIG. 4.

The control unit 106 receives the contrast effect measurements from the contrast effect measuring unit 104. In one embodiment, the control unit 106 then determines the desired infusion rate (for example, comparing the measured contrast effect with the desired contrast effect C_{target} and based on the current infusion rate), and sends control data including the desired infusion rate to the infusion unit 102. In one embodiment, the control unit 106 and the infusion unit 102 are connected by an RS-232 cable. The control unit 106 can communicate with the infusion unit 102 by other means in some embodiments, such as by optical cable, telephone line, radio signals, and so forth. The infusion unit 102 receives the control data and infuses contrast agent to the patient 110 according to the desired infusion rate.

In one embodiment, based on the contrast effect measurements, the control unit 106 determines the proper adjustment to be made to the current infusion rate, i.e., the amount of increase or decrease to the current infusion rate. For example, the control unit 106 determines whether the measured contrast effect is higher or lower than the desired contrast effect C_{target}, and selects R_{inc} or R_{inc} as the adjustment to the infusion rate. The control unit 106 sends control data including the desired infusion rate adjustment to the infusion unit 102. The infusion unit 102 receives the control data and increases or decreases its infusion rate according to the received control data.

In one embodiment at the start of the infusion process, the infusion unit 102 receives the programmed parameter Rᵢₙᵢₜ from the programming interface and begins infusion at the programmed initial infusion rate parameter Rᵢₙᵢₜ.

The ultrasound imaging unit 108 is connected to the patient 110 and performs ultrasound imaging on the patient 110. For example, a cardiac ultrasound (echocardiograph) imaging device can be used. Other types of ultrasound imaging devices can also be used. With the contrast effect measuring unit 104 measuring the contrast effect in the patient 110, the control unit 106 determining the desired infusion rate or the desired infusion rate adjustment, and the infusion unit 102 infusing contrast agent according to the determination made by the control unit 106, the ultrasound imaging unit 108 is likely to produce high quality images with a long duration of desired contrast effect and desired signal enhancement.

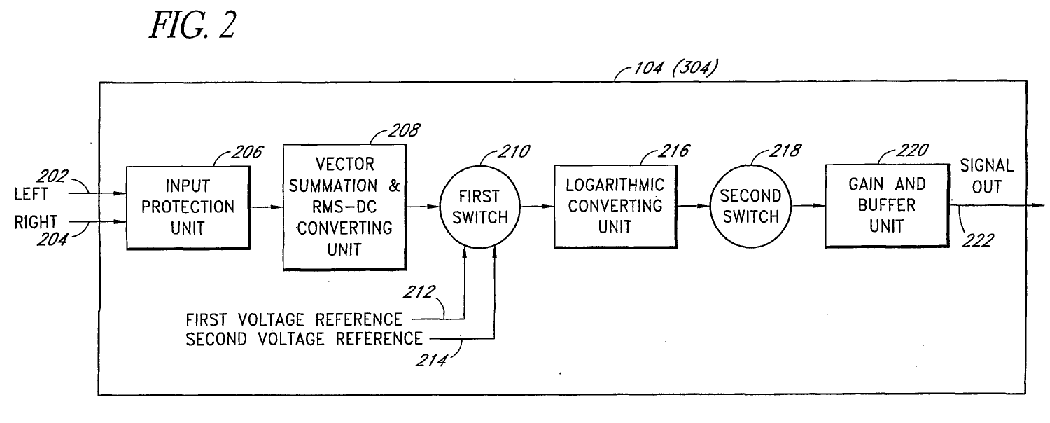

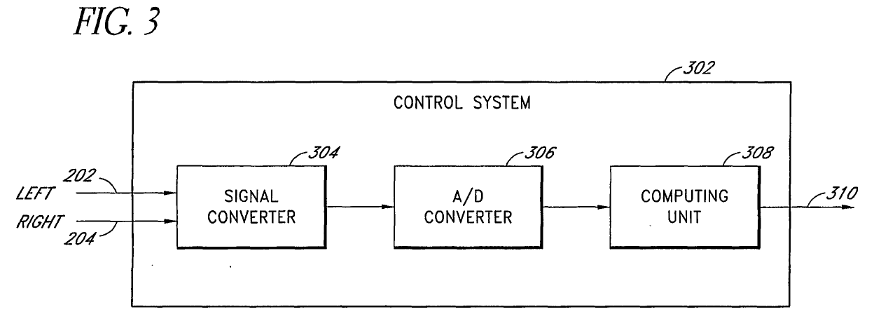

FIG. 2 shows one embodiment of a contrast effect measuring unit. FIG. 2 can also be one embodiment of the signal converter 304, which is described below in connection with FIG. 3.

The components 206, 208, 210, 216, 218 and 220 of FIG. 2 can be commercially available products or custom made. In the embodiment shown in FIG. 2, the contrast effect measuring unit 104 is a Doppler unit. The measuring unit 104 is connected to a patient 110 on one end, for example connected to a peripheral artery of the patient 110. In one embodiment, the left channel 202 receives the audio signal for the forward blood flow of the artery, and the right channel 204 receives the audio signal for the backward blood flow of the artery. In another embodiment, the left channel 202 receives the audio signal for the backward blood flow of the artery, and the right channel 204 receives the audio signal for the forward blood flow of the artery. In yet another embodiment, only one of the two channels 202 and 204 is connected to the measuring unit 104. The other channel is not connected.

The left channel 202 and the right channel 204 are connected to an input protection unit 206 on the other end. The input protection unit 206 provides over-voltage protection from erroneous or noise signals. One example of the input protection unit 206 is a clamping diode. The input protection unit 206 connects to the vector summation and RMS-DC converting unit 208.

The vector summation and RMS-DC converting unit 208 performs a vector summation process to combine the audio signals received from the left channel 202 and the right channel 204. For example, a commercially available power combiner can be used to combine the signals. In the embodiment where only one of the channels 202 and 204 is connected to the measuring unit 104, the vector summation process is not performed. The unit 208 also performs a RMS-DC converting process to convert the variant signals to constant signals. For example, one or more capacitors can be used to average the signal variations caused by cardiac cycles. A commercially available rectifier can be used to perform the conversion to direct current form. The term “RMS-DC converting” as used in the present application refers to converting signals with variances to direct current signals, which may or may not involve root mean square calculations.

The vector summation and RMS-DC converting unit 208 connects to a first switch 210, which is also connected to a first voltage reference 212 and a second voltage reference 214. The first switch 210 can be a mechanical switch or a semiconductor switch. The first voltage reference 212 and the second voltage reference 214 can be used for internal calibration. By activating the first switch 210, a user can select between the normal operation mode and the internal calibration mode.

The first switch 210 also connects to a logarithmic converting unit 216, which is connected to a second switch 218. The logarithmic converting unit 216 can convert data from linear scale to logarithmic scale. The second switch 218 can be a mechanical switch or a semiconductor switch. By activating the second switch 218, a user can select to use or not use the logarithmic converting unit 216.

The second switch 218 connects to a gain and buffer unit 220, which is connected to a signal out line 222 to send out signals. Since the measuring unit 104 may drive different units (for example an infusion unit 102 or a chart plotter) at different power (voltage) levels and different load (amp) levels, the gain and buffer unit 220 provides proper voltage gain and buffering.

In one instance of operation, Doppler audio signals from the left channel 202 and/or the right channel 204 enter through the input protection unit 206, are processed by the vector summation and RMS-DC converting unit 208, optionally converted by the logarithmic converting unit 216, and enters the gain and buffer unit 220. The gain and buffer unit 220 provides proper voltage gain and buffering, and sends the data to an infusion unit 102, a control unit 106, or a chart plotter.

FIG. 3 shows one embodiment of a control system. The control system 302 of FIG. 3 includes a signal converter 304, an AID converter 306, and a computing unit 308. The units 304, 306 can be commercially available products or custom made. One model of a commercial signal converter is Model-AD637 from Analog Devices of Norwood, Mass. One model of a commercial AID converter is Model-LTC1298 from Parallax, Inc. of Rocklin, Calif. The computing unit 308 can be custom made or modified from a commercially available product. One model of a commercial computing unit is Parallax BS2-IC from Parallax, Inc. of Rocklin, Calif. The units of the control system 302 can be combined into fewer units or separated into more units to achieve substantially the same functions. In one embodiment, the control system 302 also includes an infusion unit 102.

Referring to FIG. 3, the left channel 202 and the right channel 204 are connected to the measuring unit 104 (which is in turn connected to a patient 110) on one end, and connected to the signal converter 304 on the other end. In one embodiment, only one of the two channels 202 and 204 is connected to the measuring unit 104. The left channel 202 and/or the right channel 204 measure the Doppler audio signals of the patient's blood flow.

The signal converter 304 performs vector summation and RMS-DC conversion on the received signals. In one embodiment, the signal converter 304 also performs logarithmic conversion. In one embodiment, the signal converter 304 also performs signal scaling. For example, in one embodiment where the incoming signals from the channels 202 and 204 are in negative-to-zero voltage range but the computing unit 308 only accepts signals in zero-to-positive voltage range, the signal converter 304 scales the negative-to-zero voltage signals to zero-to-positive voltage signals. The signal converter 304 sends the processed signals to the A/D converter 306. The A/D converter 306 receives the signals and converts the signals from analog into digital format, and sends the digital signals to the computing unit 308.

The computing unit 308 receives programmed parameters from a programming interface (not shown), compares the received signals with programmed parameters, and determines a desired infusion rate or a desired infusion rate adjustment based on the received signals and the programmed parameters. For example, the computing unit 308 compares the measured contrast effect with the programmed desired contrast effect parameter, and determines a desired infusion rate or a desired infusion rate adjustment.

Some of the commercially available infusion pumps include a computing unit that accepts user-entered infusion rate and syringe size, and infuses a patient according to the entered infusion rate and syringe size. Such a commercial infusion pump and its computing unit can be modified to become the infusion unit 102 and the computing unit 308. In one embodiment, a Model-210 infusion pump from kdScientific Inc. of New Hope, Penn. was modified. Other commercial infusion pump manufacturers include Harvard Apparatus of Boston, Mass. The computing unit 308 can also be custom made. A commercial infusion pump without a computing unit can also be modified to communicate with a computing unit 308.

Referring to FIG. 3, the computing unit 308 sends the calculated desired infusion rate through output line 310 to the infusion unit 102. One example of the output line 310 is an RS-232 cable. The computing unit 308 can also be connected to the infusion unit 102 by other means, such as by optical cable, telephone line, radio signals, and so forth. The control system 302 also includes a programming interface for programming parameters, to be described below in connection with FIG. 4.

In one embodiment, the computing unit 308 is a microprocessor with programmable ROM (PROM). A personal computer can be connected to the computing unit 308 to download a program to the PROM. The computing unit 308 then uses the program to perform its functions, including receiving data from the A/D converter 306, determining desired infusion rate or adjustment to infusion rate, and sending control commands through output line 310 to the infusion unit 102. The personal computer can be disconnected from the computing unit 308 once the program is downloaded. Compared to a personal computer, the microprocessor with PROM has size and cost advantages. For example, such a microprocessor can be the size of a stamp or smaller, and cost about $100 or less.

FIG. 4 shows one embodiment of a programming interface for programming parameters. In one embodiment, the programming interface 402 is included in a control unit 106. In another embodiment, the programming interface 402 is included in a control system 302. A user uses the programming interface 402 to program parameters.

Referring to FIG. 4, the programming interface 402 includes a first face 404 for programming the desired contrast effect parameter C_{target}. By moving a first knob 406 along the first face 404, a user can set the desired contrast effect parameter to be any value between 0 and 40 dB. The programming interface 402 also includes a second face 414 for programming the increment to infusion rate parameter R_{inc} and a third face 424 for programming the decrement to infusion rate parameter R_{dec}. The second face 414 and the third face 424 includes a second knob 416 and a third knob 426, respectively, for setting the R_{inc} and R_{dec} parameter to be any value between 0 and 0.4 cc/minute, respectively.

In one embodiment, the faces 404, 414, 424 and the knobs 406, 416, 426 are located on a panel of the computing unit 308. In another embodiment, push buttons are located on a panel of the computing unit 308 for programming the parameters. For example, a push button labeled “increase desired contrast effect” and a push button labeled “decrease desired contrast effect” can be used to program the desired contrast effect parameter. Each time the “increase desired contrast effect” button or the “decrease desired contrast effect” button is pushed, the computing unit 308 respectively increases or decreases the desired contrast effect parameter by a pre-set value, for example 1 dB. A display device can be connected to the computing unit 308 to display the parameter value currently being programmed. Although FIG. 4 shows knobs 406, 416 and 426 for ease of illustration, it should be understood that where the computing unit 308 is a microprocessor, it is often easier to program a microprocessor to read data from buttons than from knobs.

In other embodiments, users program parameters using a personal computer connected to the control unit 106 or connected to the computing unit 308. For example, users use a keyboard, a keypad, a mouse, or a touch-screen connected to the personal computer to program parameters. Devices can be connected by communications cables or wires or by radio signals. A monitor of the personal computer can display output to the user, for example showing the currently programmed parameter values, the currently measured contrast effect, and the current infusion rate. A display device of the control unit 106 or of the computing unit 308 can also display such output. After the parameters are programmed, they can be downloaded from the personal computer into the control unit 106 or into the computing unit 308. The personal computer can also function as the computing unit 308 itself.

In one embodiment, the infusion rate adjustment parameters are programmed by the manufacturer of the programming interface 402 and cannot be adjusted by a user. In another embodiment, these parameters can be adjusted by a user.

In one embodiment, more than one desired contrast effect parameters are used. For example, a parameter for optimal contrast effect and a parameter for reasonable variation are used. If the measured contrast effect falls within the reasonable variation from the optimal contrast effect, then no adjustments are made to the current infusion rate of the infusion unit 102. Otherwise an adjustment is made to the current infusion rate. In another example, two parameters represent the lower limit and the higher limit of the desired contrast effect, respectively. If the measured contrast effect falls within the range of the two limits, then no adjustments are made to the current infusion rate of the infusion unit 102. Otherwise an adjustment is made to the current infusion rate.

Other embodiments of programmed parameters can also be used. For example, when the measured contrast effect is lower than the desired contrast effect C_{target}, two programmable parameters R_{inc1} and R_{inc2} can be used. When the measured contrast effect is slightly lower than C_{target}, R_{inc1} (for example programmed with a value of 0.1-cc/minute) is used. When the measured contrast effect is significantly lower than C_{target}, R_{inc2} (for example programmed with a value of 0.2-cc/minute) is used. Similarly, more than one R_{dec} parameters can also be used when the measured contrast effect is higher than the desired contrast effect C_{target}. In another example, a user programs a fast infusion rate R_{fast} to be used when the measured contrast effect is lower than C_{target}, and a slow infusion rate R_{slow} to be used when the measured contrast effect is higher than C_{target}. However, it takes some time for a micro-bubble concentration to reach equilibrium in a patient's body, and the contrast effect measured by the measuring unit 104 may not be equal to the contrast effect at the patient's body region where the ultrasound imaging is conducted. Moreover, a rapid change in infusion rate may reduce the duration of the desired contrast effect. Therefore it is often preferable to slowly adjust the infusion rate at small increments or small decrements.

In other embodiments, the control unit 106 or the control system 302 calculates the infusion rate or the adjustment to infusion rate, without using parameters R_{inc}, R_{dec}, R_{fast} or R_{slow}. For example, the control unit 106 or the control system 302 uses a formula to calculate an infusion rate based on the difference between the measured contrast effect and a programmed desired contrast effect parameter. Using the formula for calculation, if the measured contrast effect is lower than the desired contrast effect, then the formula calculates a higher infusion rate. If the measured contrast effect is higher than the desired contrast effect, then the formula calculates a lower infusion rate. The higher and lower infusion rates are made somewhat proportional to the difference between the measured contrast effect and the desired contrast effect parameter. A formula such as Rᵢₙᵢₜ+c*(C_{target}−Cₘₑₐₛᵤᵣₑ) can be used to determine a desired infusion rate, with c being a constant and Cₘₑₐₛᵤᵣₑ being the measured contrast effect. However, as described in the paragraph above, it is often preferable to slowly adjust the infusion rate at small increments or small decrements. Therefore it may be preferable to limit the calculated infusion rate to a small deviation from the initial or current infusion rate. The higher and lower infusion rates can also be pre-set values determined by the manufacturer of the control unit 106 or the control system 302.

In one embodiment, the programming interface 402 is connected to the ultrasound imaging unit 108. The ultrasound imaging unit 108 receives the programmed desired contrast effect parameter from the programming interface 402, and displays or stores the parameter along with the ultrasound imaging results. Therefore a user administering a follow-up ultrasound imaging test can review the previous ultrasound imaging results, and program the desired contrast effect parameter to be the same value as the last test. Since the tests are taken with the same contrast effect, they are likely to have the same level of signal enhancement and are thus ideal for comparisons. Instead of having the ultrasound imaging unit 108 automatically displaying or storing the desired contrast effect parameter, a user can also record the parameter, for example on a piece of paper, on a ultrasound imaging chart, or into a computer.

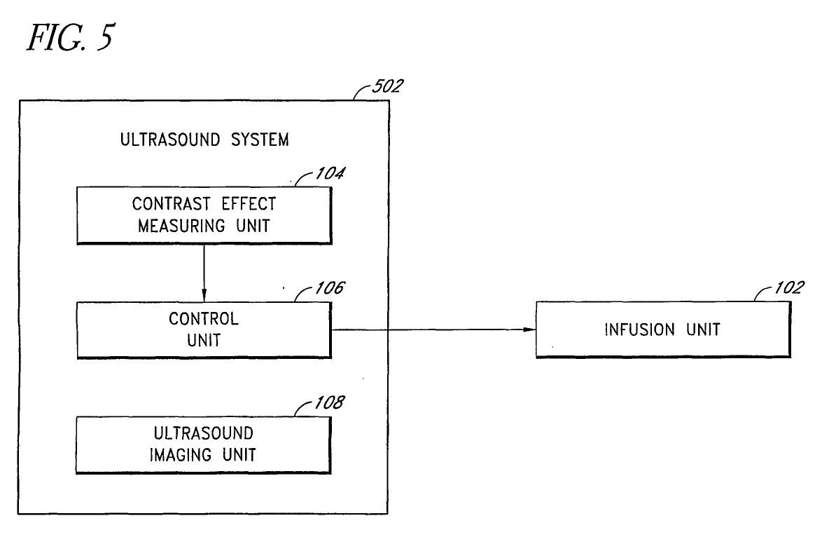

FIG. 5 shows one embodiment of an ultrasound system. The ultrasound system 502 of FIG. 5 includes a contrast effect measuring unit 104 and a control unit 106. In one embodiment, the ultrasound system 502 also includes the ultrasound imaging unit 108. In another embodiment, the ultrasound imaging unit 108 is not included within the ultrasound system 502.

The contrast effect measuring unit 104 measures the contrast effect of a contrast agent in the patient 110, and sends the measured signals to the control unit 106. In one embodiment, the ultrasound system 502 is a commercial ultrasound system that includes a commercial Doppler frequency measuring unit. The Doppler frequency measuring unit measures the Doppler signal and uses the Doppler frequency shift in a patient's artery or vein to determine the direction and velocity of blood flow. With the Doppler signal measured by the Doppler frequency measuring unit, its signal intensity is used to measure the effect of the contrast enhancement.

The control unit 106 receives the signals and determines the desired infusion rate or adjustment to the infusion rate. The control unit 106 sends the desired infusion rate or adjustment to infusion rate to the infusion unit 102. The infusion unit 102 receives the desired infusion rate or adjustment to infusion rate from the control unit 106 and infuses the patient 110 according to the desired infusion rate, or changes its infusion rate according to the received adjustment to infusion rate.

In one embodiment, the control unit 106 is connected to the ultrasound imaging unit 108. The ultrasound imaging unit 108 receives from the control unit 106 the measured contrast effect. The ultrasound imaging unit 108 displays or stores the measured contrast effect along with the ultrasound imaging results. In another embodiment, the ultrasound imaging unit 108 displays or stores the programmed desired contrast effect parameter along with the ultrasound imaging results. Therefore, follow-up ultrasound imaging tests can be taken that achieve the same contrast effect. In yet another embodiment, a person administering the ultrasound imaging records the programmed desired contrast effect parameter or the measured contrast effect along with the ultrasound imaging results, and follow-up ultrasound imaging tests can be taken with the same contrast effect.

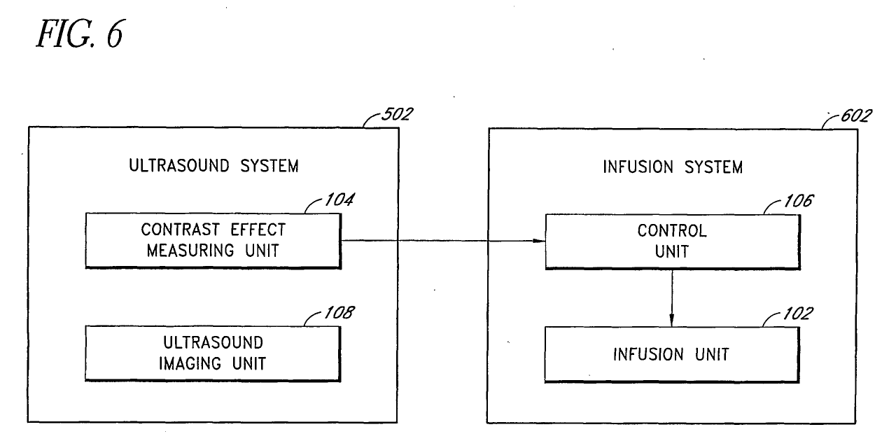

FIG. 6 shows one embodiment of an ultrasound system and one embodiment of an infusion system. In FIG. 6, the ultrasound system 502 includes a contrast effect measuring unit 104. The infusion system 502 includes a control unit 106 and an infusion unit 102. In one embodiment, the contrast effect measuring unit 104 is included in the infusion system 502. One embodiment of the ultrasound system 502 also includes the ultrasound imaging unit 108. As described above in connection with FIG. 5, the ultrasound system 502 can be modified from a commercial ultrasound system that includes a commercial Doppler frequency measuring unit.

Referring to FIG. 6, the contrast effect measuring unit 104 measures the contrast effect of a contrast agent in the patient 110, and sends the measured signals to the control unit 106 of the infusion system 602. The control unit 106 receives the signals and determines the desired infusion rate or adjustment to infusion rate. The control unit 106 sends the desired infusion rate or adjustment to infusion rate to the infusion unit 102 of the infusion system 602. The infusion unit 102 receives the desired infusion rate from the control unit 106 and infuses the patient 110 according to the desired infusion rate, or receives the desired adjustment to infusion rate and adjusts its infusion rate accordingly.

In one embodiment, the contrast effect measuring unit 104 is connected to the ultrasound imaging unit 108. The ultrasound imaging unit 108 receives from the contrast effect measuring unit 104 the measured contrast effect. The ultrasound imaging unit 108 displays or stores the measured contrast effect along with the ultrasound imaging results. In another embodiment, the ultrasound imaging unit 108 displays or stores the programmed desired contrast effect parameter along with the ultrasound imaging results. Therefore, follow-up ultrasound imaging tests can be taken to achieve the same contrast effect. In yet another embodiment, a person administering the ultrasound imaging records the measured contrast effect or the programmed desired contrast effect parameter along with the ultrasound imaging results, and follow-up ultrasound imaging tests can be taken with the same contrast effect.

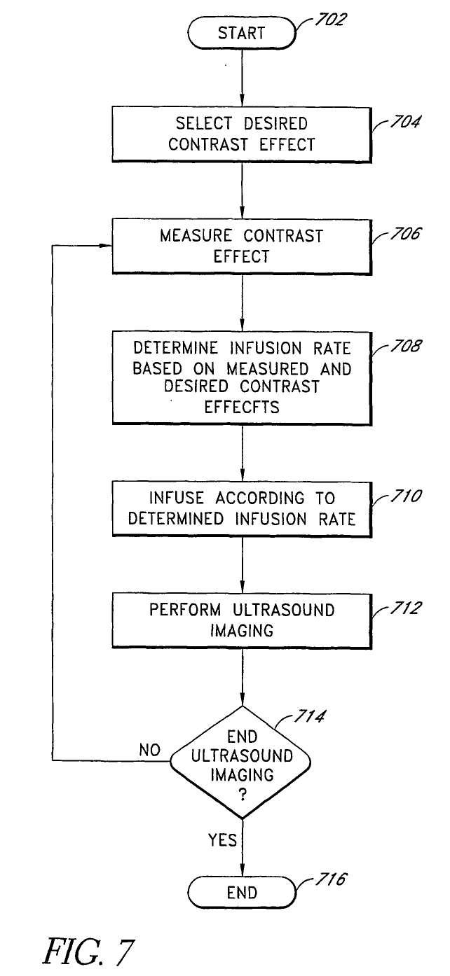

FIG. 7 shows one embodiment of a process of delivering and controlling contrast agent. From a start block 702, the process proceeds to a block 704. At the block 704, the programming device 402 allows a user to program one or more desired contrast effect parameters. In some embodiments, programmable parameters may also include initial infusion rate, increment adjustment to infusion rate, and decrement adjustment to infusion rate. The process proceeds to a block 706, where the contrast effect measuring unit 104 measures the contrast effect in a patient 110. The process proceeds to a block 708.

At the block 708, the control unit 106 determines a desired infusion rate or adjustment to infusion rate, based on the contrast effect measured by the measuring unit 104 and the desired contrast effect parameter entered from the programming interface 402. The process proceeds to a block 710, where the infusion unit 102 infuses contrast agent into the patient 110 according to the determined desired infusion rate or adjustment to infusion rate. In one embodiment, the infusion unit 102 first begins infusing at a programmed initial infusion rate. The process proceeds to a block 712, where the ultrasound imaging unit 108 performs ultrasound imaging on the patient 110.

The process proceeds to a decision block 714. At the block 714, a determination is made as to whether the ultrasound imaging test is ending. If the imaging test is still in progress, then the process returns to the block 706, to continue measuring contrast effect in the patient. If the imaging test is ending, then the process proceeds to an end block 716.

In one embodiment, the measuring of block 706 and the determining of block 708 may end before the ultrasound imaging test ends. For example, after the measured contrast effect has been steady for a period of time or after the infusion unit 102 has been infusing at a steady rate for a period of time, a user may decide to stop measuring contrast effect or stop adjusting the infusion rate, and allow the infusion rate to remain unchanged until the end of the ultrasound imaging test. The period of time can be a programmed parameter or a value pre-set in the measuring unit 104, in the control unit 106, or in the control system 302.

The disclosed methods and systems can be used to monitor the progress of drug delivery or gene therapy. In one embodiment, an ultrasound contrast agent is bound to a drug. The bound drug/contrast agent compound is infused into the patient to reach a target tissue. The contrast effect measuring unit 104 measures the contrast effect of the compound to provide information as to the flow and delivery rate of the drug/contrast agent compound inside the patient, and whether the compound has reached the target tissue. To control the infusion rate of the drug/contrast agent compound, the control unit 106 determines the desired infusion rate or the desired infusion rate adjustment based on the measured contrast effect and programmed parameters. The infusion unit 102 adjusts infusion rate based on the control unit's determination. The methods and systems can also be applied to a gene/contrast agent bound together.

While certain embodiments of the inventions have been described, these embodiments have been presented by way of example only, and are not intended to limit the scope of the inventions. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A system for controlling delivery of a contrast agent for ultrasound imaging, the system comprising:
a contrast effect measuring unit configured to measure a contrast effect of an ultrasound contrast agent in the blood or an organ of a patient;
a control unit configured to determine a contrast agent infusion rate based on the measured contrast effect; and
an infusion unit configured to infuse the patient with the ultrasound contrast agent at the determined contrast agent infusion rate.

2. The system of claim 1, further comprising a display unit configured to display the measured contrast effect.

3. The system of claim 1, wherein the contrast effect comprises an ultrasound or Doppler signal in a blood vessel of the patient.

4. The system of claim 1, wherein the control unit is configured to convert analog signals to digital signals.

5. The system of claim 1, further comprising a programming interface for programming one or more desired contrast effect parameters, wherein the control unit is configured to determine a contrast agent infusion rate based on comparing the measured contrast effect with the programmed desired contrast effect parameters.

6. The system of claim 5, further comprising a programming interface for programming one or more infusion rate adjustment parameters, wherein the control unit is configured to determine a contrast agent infusion rate based on comparing the measured contrast effect with the programmed desired contrast effect parameters, and based on the programmed infusion rate adjustment parameters.

7. A method of determining the infusion rate for delivery of a contrast agent for, ultrasound imaging, the method comprising:
measuring a contrast effect of an ultrasound contrast agent in the blood or an organ of a patient; and
automatically determining an infusion rate of the ultrasound contrast agent based on the measured contrast effect.

8. The method of claim 7, further comprising performing an ultrasound imaging test on the patient.

9. The method of claim 8, wherein measuring the contrast effect occurs continuously during at least part of a duration of the ultrasound imaging test.

10. The method of claim 7, wherein measuring the contrast effect comprises measuring an intravascular micro-bubble concentration of the ultrasound contrast agent in the patient.

11. The method of claim 7, wherein the contrast effect comprises an ultrasound or Doppler signal in a blood vessel of the patient.

12. The method of claim 7, further comprising prompting a user to program one or more desired ultrasound or Doppler signal parameters into a system that automatically determines the contrast agent infusion rate, and wherein determining the contrast agent infusion rate is based on the measured contrast effect and based on the programmed desired ultrasound or Doppler signal parameters.

13. The method of claim 7, further comprising prompting a user to program at least one parameter into a system that automatically determines the contrast agent infusion rate, and wherein determining a contrast agent infusion rate is based on the measured contrast effect and based on the at least one parameter.

14. A system for controlling the delivery of contrast agent for ultrasound imaging, the system comprising:
a programming interface configured to accept input of one or more desired contrast effect parameters and one or more infusion rate adjustment parameters;
a measuring unit configured to measure a contrast effect of an ultrasound contrast agent in a patient during at least part of a duration of an ultrasound imaging test on the patient;
a control unit configured to receive information regarding the measured contrast effect from the measuring unit, to make a comparison between the measured contrast effect and the desired contrast effect parameters, to generate an infusion rate based on the comparison, and to send information regarding the infusion rate to an infusion unit, wherein the infusion unit is configured to receive the information regarding the infusion rate and to infuse a contrast agent into the patient at the infusion rate.

15. A system for controlling the delivery of contrast agent for ultrasound imaging, the system comprising:
a programming interface configured to program one or more desired contrast effect parameters and to program one or more infusion rate parameters;
a measuring unit configured to continuously measure contrast effect of an ultrasound contrast agent in a patient during at least a partial duration of performing an ultrasound imaging test on the patient;
a control unit configured to receive the measured contrast effect from the measuring unit, to compare the measured contrast effect with the programmed contrast effect parameters, to select an infusion rate parameter based on the comparison, and to send the selected infusion rate parameter to an infusion unit, wherein the infusion unit is configured to receive the selected infusion rate parameter and to infuse the patient according to the received infusion rate parameter.

16. A system for controlling the delivery of contrast agent for ultrasound imaging, the system comprising:
a measuring means for measuring contrast effect of an ultrasound contrast agent in a patient and producing signals representing the measured contrast effect;
a controlling means for determining a contrast agent infusion rate based on the produced signals and based on one or more programmed parameters; and
an infusing means for infusing the patient with the ultrasound contrast agent at the determined infusion rate.

17. The system of claim 16, further comprising a programming means for programming the programmed parameters.

18. A system for controlling the delivery of contrast agent for ultrasound imaging, the system comprising:
a programming interface configured to receive one or more programmed parameters and configured to display the programmed parameters;
a signal processing unit configured to receive an ultrasound or Doppler signal representing a contrast effect of an ultrasound contrast agent in a patient and configured to process the received signal; and
a computing unit configured to determine a contrast agent infusion rate based on the processed signal and based on the programmed parameters, and configured to send information regarding the determined infusion rate to an infusion device.

19. The system of claim 18, wherein the programming interface is configured to receive one or more desired contrast effect parameters.

20. The system of claim 18, wherein the signal processing unit is configured to process the received signals by performing vector summation.

21. The system of claim 18, wherein the signal processing unit is configured to process the received signals by performing RMS-DC conversion.

22. The system of claim 18, wherein the signal processing unit is configured to process the received signals by performing logarithmic conversion.

23. The system of claim 18, wherein the signal processing unit is configured to process the received signals by converting the received signals from analog form to digital form.

24. The system of claim 18, further comprising an infusion device configured to receive the information regarding the determined infusion rate sent from the computing unit, and configured to infuse the patient with the ultrasound contrast agent at the determined infusion rate.

25. The system of claim 18, further comprising a measuring unit configured to measure contrast effect and to send an audio signal representing the measured contrast effect to the signal processing unit.
